(19) United States
(12) Patent Application Publication
(10) Pub. No.: US 2022/0283254 A1
(43) Pub. Date: Sep. 8, 2022

(54) METHOD FOR DETERMINING A SIMULATION VALUE FOR AN MR MEASUREMENT, A COMPUTING UNIT, A SYSTEM, AND A COMPUTER PROGRAM PRODUCT

(71) Applicant: Siemens Healthcare GmbH, Erlangen (DE)

(72) Inventors: Matthias Gebhardt, Erlangen (DE); Mario Zeller, Erlangen (DE)

(21) Appl. No.: 17/687,613

(22) Filed: Mar. 5, 2022

(30) Foreign Application Priority Data
Mar. 5, 2021 (EP) 21161008.4

(51) Int. Cl. G01R 33/54; G01R 33/28; A61B 5/055

(52) U.S. Cl. CPC G01R 33/543; G01R 33/288; A61B 5/055

(57) ABSTRACT

A method for determining a simulation value describing a safety-related variable for an MR measurement includes providing an MR pulse sequence that is configured to perform an MR measurement of a patient using an MR scanner based on the MR pulse sequence. The MR pulse sequence includes a temporal succession of RF pulses and gradient pulses. A patient value describing a characteristic of the patient is provided. Based on the MR pulse sequence and the patient value, the simulation value is determined by a computing unit. The simulation value describes a safety-relevant variable for performing an MR measurement using the MR pulse sequence. For determining the simulation value, specific characteristics of the RF pulses and of the gradient pulses of the MR pulse sequence as well as a temporal succession of the RF pulses and the gradient pulses are taken into account.

This application claims the benefit of European Patent Application No. EP 21161008.4, filed on Mar. 5, 2021, which is hereby incorporated by reference in its entirety.

### BACKGROUND

The present embodiments relate to a method for determining a simulation value describing a safety-relevant variable for an MR measurement, a computing unit, a system, and a computer program product.

In medical technology, imaging by magnetic resonance (MR) (e.g., magnetic resonance tomography (MRT) or magnetic resonance imaging (MRI)) is characterized by high soft tissue contrasts. A human or animal patient is typically positioned in the examination space of an MR scanner. During an MR measurement, radiofrequency (RF) pulses are typically radiated into the object under examination using a radiofrequency antenna unit of the MR scanner. The RF pulse generates an alternating magnetic field (e.g., a B1 field) in the examination space. This is distinct from a static main magnetic field (e.g., the B0 field). In addition, gradient pulses are switched using a gradient coil unit of the MR scanner, causing temporary magnetic field gradients to be generated in the examination space. The pulses generated excite and trigger spatially-encoded MR signals in the patient. The MR signals are received by the MR scanner and used to reconstruct MR images.

For operating MR scanners, various normative requirements regarding a specific absorption rate (SAR) and/or a B1+rms value are usually to be met when applying the RF pulses and/or stimulating the patient by switching of the gradient pulses. If the patient has an implant, particularly stringent requirements usually have to be met. However, to also protect critical components of the MR scanner, such as RF amplifiers, for example, it may be necessary to limit the power of the RF irradiation and/or the variation over time of the currents flowing through the gradient coil unit in order to prevent excessive component heating.

Common safety architectures include real-time monitoring of measured variables determined during the MR measurement that correlates with the transmit activity of the RF antenna unit and/or the activity of the gradient coil unit. If a limit is exceeded, the MR measurement is then automatically aborted. However, such aborts may remain the exceptional case in clinical operation, as such aborts do not merely cause frustration to the patients and the operators of the MR scanner. For example, such aborts may result in pointless invasive procedures, while making it impossible to repeat the MR measurement immediately afterwards (e.g., in the case of contrast agent administration because of the contrast agent absorbed in the patient's tissue).

### SUMMARY AND DESCRIPTION

The scope of the present invention is defined solely by the appended claims and is not affected to any degree by the statements within this summary.

The present embodiments may obviate one or more of the drawbacks or limitations in the related art. For example, such scanning aborts may be minimized or aborted.

A method for determining a simulation value describing a safety-relevant variable for a magnetic resonance (MR) measurement is provided. An MR pulse sequence that is configured as the basis for performing an MR measurement of a patient using an MR scanner is provided. The MR pulse sequence includes a temporal succession of RF pulses and a plurality of gradient pulses. In addition, at least one patient value describing a characteristic of the patient is provided. In the case of a plurality of patient values, for example, each of these patient values may describe a respective characteristic. Such a characteristic may be, for example, the patient's weight, height, age, or gender. Such a characteristic may relate to spatial dimensions of the patient's anatomy in the examination space (e.g., captured in advance by a 3D camera) and a relative distribution of muscles and fat in the examination space. Another possible patient value may also relate, for example, to whether the patient has an implant. In addition, the at least one patient value may describe the characteristic of the patient's position in the MR scanner (e.g., how the patient is positioned in the examination space, such as whether the patient is positioned head or foot first in the examination space).

Based on the MR pulse sequence and the at least one patient value, at least one simulation value is determined by a computing unit. The at least one simulation value describes a safety-relevant variable for performing an MR measurement using the MR pulse sequence. For determining the at least one simulation value, specific characteristics of the RF pulses (e.g., of all the RF pulses) and of the gradient pulses (e.g., of all the gradient pulses) of the MR pulse sequence as well as the temporal succession thereof are taken into account. The at least one simulation value is also made available.

The MR pulse sequence may be provided, for example, by a first interface. The at least one simulation value may be provided, for example, by a second interface. The MR pulse sequence and/or the at least one simulation value may be provided, for example, in the form of a dataset.

The MR pulse sequence provided is configured, for example, as the basis for performing a complete MR measurement of a patient using an MR scanner. The MR pulse sequence may include and/or describes all the RF pulses and all the gradient pulses that are applied or switched during the MR measurement. The MR pulse sequence may include all the information to be provided for defining the desired RF pulses and all the gradient pulses of the MR pulse sequence that will be used during the MR measurement. The MR measurement may be suitable for acquiring MR signals from which at least one MR image (e.g., two-dimensional MR image) be reconstructed.

For example, the safety-relevant variable may relate to the safety of the patient and/or the safety of the MR scanner. The safety-relevant variable may be used to infer a risk of the patient and/or the MR scanner being harmed/damaged if the MR scanner were to use or rather play out the MR sequence.

For example, the specific characteristics to be considered in determining the at least one simulation value may include the shape and/or duration and/or amplitude of the RF pulses and/or of the gradient pulses. Determining the at least one simulation value may involve unrolling the MR pulse sequence. The unrolling of the MR pulse sequence may involve complete simulation of the MR pulse sequence (e.g., taking all the RF pulses and all the gradient pulses into account). The unrolling may be based on raw information of the MR pulse sequence and not, for example, on any already compressed and/or derived variables of the MR pulse sequence. For example, the unrolling takes into account the specific shape and/or duration and/or amplitude of each of the RF pulses and/or gradient pulses, as well as the time intervals between the pulses. These may be considered not only for a sub-section of the MR pulse sequence, but for the entire MR pulse sequence.

Determining the at least one simulation value in this way (e.g., by unrolling the MR pulse sequence) may obviate the need to develop any methods specifically geared to the respective MR pulse sequence type for rapid preliminary determination of patient exposure to RF pulses and/or gradient pulses, as is usually the case in the prior art. Often, such methods are developed essentially independently of the actual MR sequence, which provides that the same data is not necessarily accessed. Rather, the developer of the MR pulse sequence is to make the most reasonable “worst case” estimate possible. In a worst case scenario, an estimate that is not chosen to be restrictive enough will result in the measurement being aborted; an overly conservative estimate will result in the available power of the MR system not being accessed, which may result in an unnecessarily long measurement time and/or reduced image quality. In one embodiment, the method of one or more of the present embodiments enables such disadvantages of the prior art to be overcome.

The at least one simulation value may be determined and provided in real time. Here, “real time” may be a period of time that is short enough that determining the at least one simulation value does not prolong and/or impede the course of the MR measurement (e.g., in a noticeable manner for the patient and/or an operator). In one embodiment, the determination of the at least one simulation value runs in the background.

The at least one simulation value may describe a specific absorption rate (SAR) and/or a gradient stimulation.

The SAR may describe the radiofrequency energy absorbed per unit time and patient mass through application of the RF pulses. The absorption of RF energy may result in heating of the patient's body tissue. Energy absorption is an important variable for setting safety limits. In the event of an impermissibly high local concentration of RF energy, RF burns may occur (e.g., local SAR). If the RF energy is evenly distributed over the entire body, the stress on thermoregulation or rather the patient's cardiovascular system is crucial (e.g., whole-body SAR). The SAR may be achieved, for example, by low energy RF pulses, smaller flip angles, shorter repetition time (TR), and/or by measuring fewer slices.

For example, gradient stimulation may include stimulation of the patient's nerves. For example, gradient stimulation may include peripheral nerve stimulation (PNS). Time-varying magnetic fields may be used to induce electrical currents in the patient's body and stimulate nerves or muscles. This stimulation may be perceived as uncomfortable by the patient.

The method may also include comparing the at least one simulation value with a predefined limit value, and, if the at least one simulation value does not exceed the predefined limit value, performing an MR measurement on the patient using the MR scanner based on the MR pulse sequence. This may check whether the patient and/or the MR scanner would be harmed/damaged by performing the MR measurement according to the MR sequence, and only if this is not the case would the MR measurement be performed according to the MR sequence.

One embodiment of the method provides that the determination of the at least one simulation value is performed by a non-local computing unit.

The non-local computing unit may be located at a different location from that of the MR scanner. For example, the non-local computing unit is not located in the same room and/or in an adjacent room and/or in the same building as the MR scanner. The non-local computing unit may be based on an IT infrastructure provided via a computer network, without the IT infrastructure being installed on a local computer of the MR scanner. The non-local computing unit may be based on cloud computing and/or an IT infrastructure that is provided for example via the Internet.

The non-local computing unit may include high-performance computers that are configured to determine the at least one simulation value in real time.

Another embodiment of the method provides that the computing unit includes a database, where the database contains descriptions of a plurality of MR pulse sequence types. At least one MR pulse sequence type ID is provided to the computing unit, where the at least one MR pulse sequence type ID is assigned to one MR pulse sequence type of the plurality of MR pulse sequence types. At least one MR pulse sequence parameter is provided to the computing unit. The MR pulse sequence is determined by the computing unit based on the at least one MR pulse sequence type ID and the at least one MR pulse sequence parameter.

In one embodiment, by holding and/or storing the plurality of MR pulse sequence types in the database, it may be achieved that only the at least one MR pulse sequence parameter is to be transmitted to the computing unit, but not the MR pulse sequence (e.g., the entire MR pulse sequence).

An MR pulse sequence type may, for example, have a type-specific structure and/or a type-specific pattern (e.g., of RF pulses and/or gradient pulses). Such a structure and/or such a pattern may, for example, include an arrangement of interacting and/or interconnected elements. Such elements may, for example, be RF pulses and/or gradient pulses.

The MR pulse sequence types may be parameterizable (e.g., by specifying the at least one MR pulse sequence parameter, an MR pulse sequence, such as a fully defined MR pulse sequence that uniquely describes a succession of RF pulses and/or gradient pulses, may be derived from an MR pulse sequence type). For example, an MR pulse sequence type may provide a framework that may be filled in by providing the at least one MR pulse sequence parameter. For example, an MR pulse sequence parameter may include a number of repetitions of a sequence section and/or a flip angle, etc.

An MR pulse sequence type may describe one or more MR pulse sequence sections (e.g., for a diffusion sequence, each different diffusion encoding constitutes a subsection, the fat saturation, and the readout module). The subsections may be parameterized separately, for example.

For example, an MR pulse sequence type ID may be a name and/or number used to designate an MR pulse sequence type.

Another embodiment of the method provides that the computing unit includes a database, where the database includes at least one pre-calculated auxiliary value. The at least one simulation value is determined using the at least one auxiliary value. For example, the at least one auxiliary value is assigned a variation of patient values and/or MR pulse sequence parameters.

For example, the database for determining at least one simulation value (e.g., an optimum SAR prediction) may include at least one pre-calculated auxiliary value for at least one MR pulse sequence type. For example, limits for variations of patient and sequence parameters may already be calculated in advance and stored as auxiliary values.

The at least one auxiliary value may be assigned to a section of the MR pulse sequence (e.g., an MR pulse sequence section). The at least one simulation value may be determined section by section for the respective MR pulse sequence sections.

The at least one auxiliary value may, for example, be based on modeling of at least one MR pulse sequence type for at least one patient value and/or for at least one MR pulse sequence parameter. The at least one auxiliary value may, for example, take into consideration a variation of a measurement time for performing the MR measurement. For example, the at least one auxiliary value may relate to modeling of the patient, a spatial scan coverage by MR signals to be acquired, and/or a range of MR signals to be acquired. For example, one or more MR pulse sequence types may have been modeled for a set of patient parameters, such as weight and/or body size, and the result of the modeling may have been stored as auxiliary values. For example, for optimum stimulation prediction, a slice orientation may have been tilted for a number of angles. In addition, for example, the effect of a measurement time lengthening (e.g., by increasing a number of averages and/or a matrix size) may be checked in advance. In one embodiment, this procedure allows, for example, a real-time prediction of setting changes on the executability of an MR sequence during editing of one of the MR sequences.

The at least one auxiliary value may relate, for example, to an MR pulse sequence section. For example, at least one auxiliary value may be calculated for an MR pulse sequence section. The MR pulse sequence sections may be quickly recalculated and/or combined, for example, by additional parameterization describing influencing values from a preceding MR pulse sequence section (e.g., already incurred SAR, current stimulation value, etc.). For example, an MR pulse sequence may be composed of known blocks.

Another embodiment of the method provides that at least one adjustment value is provided, for example, by a third interface. In this case, the at least one simulation value is also determined by the computing unit based on the at least one adjustment value.

The at least one adjustment value may, for example, describe a characteristic (e.g., temporary) and/or an operating parameter of the MR scanner. This characteristic and/or this operating parameter may relate, for example, to an RF transmit voltage (e.g., a maximum RF amplitude) and/or a patient-dependent scaling factor that allows conversion of flip angle to RF transmit voltage, and/or a gradient offset that enables external or patient-specific magnetic field deviations to be compensated.

The at least one simulation value may be determined even more accurately using the at least one adjustment value.

Another embodiment of the method provides that a protocol queue (e.g., a protocol set) including a plurality of MR pulse sequences is provided to the computing unit, where for each MR pulse sequence of the plurality of MR pulse sequences, at least one simulation value is determined by the computing unit. Such a protocol queue may include all the MR pulse sequences measured in the course of an MR examination of a patient. For example, a localizer measurement is first performed, which is followed (e.g., automatically) by measurement planning resulting in the protocol queue. Rather than waiting until it is the turn of an MR sequence, the MR sequence may be unrolled and/or checked beforehand.

A protocol queue may include a temporal succession of a plurality of MR pulse sequences. Each of these MR pulse sequences may describe a respective MR measurement. For example, the at least one simulation value (and also a possible comparison of the at least one simulation value with a predefined limit value) may be determined for MR measurements following a current MR measurement and/or already planned MR measurements in the protocol queue. In one embodiment, possible exceedances, for example, may thus be detected at an early stage, and/or any conflicts may be resolved in good time.

Another embodiment of the method provides that the MR pulse sequence (e.g., at least one MR pulse sequence parameter) is optimized based on the simulation value. This optimization may take place, for example, using a neural network. An optimization of this kind may be performed by a non-local computing unit. For example, more complex optimization of adjustable MR pulse sequence parameters may take place on high-performance cloud computers.

In one embodiment, the optimization may take place automatically (e.g., without intervention by an operator of the MR scanner). In one embodiment, however, a suggestion may be made to an operator of the MR scanner (e.g., as part of a preview), according to which at least one MR pulse sequence parameter of the MR pulse sequence may be adjusted. The operator may, for example, reject the suggestion, accept the suggestion unchanged, or make changes to the suggestion.

The present embodiments also include a computer unit for determining at least one simulation value that is configured to determine the at least one simulation value based on an MR pulse sequence and at least one patient value. The MR pulse sequence includes a temporal succession of a plurality of RF pulses and a plurality of gradient pulses, where the at least one patient value describes a characteristic of the patient. The at least one simulation value describes a safety-relevant variable when performing an MR measurement based on the MR pulse sequence. The computing unit is further configured to take into consideration specific characteristics (e.g., the shape and/or duration and/or amplitude) of all the RF pulses and all the gradient pulses of the MR pulse sequence, as well as their temporal succession, when determining the at least one simulation value.

The advantages of the computing unit for determining the at least one simulation value essentially correspond to the advantages of a method for determining a simulation value describing a safety-relevant variable for an MR measurement, as detailed above. Features, advantages, or alternative embodiments mentioned herein may likewise be applied to the other subject matters, and vice versa.

In addition, an MR scanner with a computing unit as described above is provided.

The present embodiments also include a computer program product that includes a program and may be loaded directly into a memory of a computing unit for determining at least one simulation value and has program means (e.g., libraries and auxiliary functions) for carrying out a method according to the present embodiments when the computer program product is executed in the computing unit. The computer program product may include software with a source code that still needs to be compiled and bound or that only needs to be interpreted, or an executable software code that only needs to be loaded into the system control unit for execution. The computer program product enables the method according to the present embodiments to be executed in a fast, identically repeatable, and robust manner. The computer program product is configured such that the computer program product may execute corresponding method acts by the computing unit. The computing unit may have the requirements for efficiently carrying out the respective method acts, such as an appropriate main memory, an appropriate graphics card, or an appropriate logic unit.

The computer program product is stored, for example, on a computer-readable medium or on a network or server. For example, the computer program product may be loaded into a processor of a local system control unit that may be directly connected to an MR scanner or implemented as part of the MR scanner.

In addition, control information of the computer program product may be stored on an electronically readable data carrier. The control information of the electronically readable data carrier may be configured to carry out a method according to the present embodiments when the data carrier is used in a computing unit. Examples of electronically readable data carriers are a DVD, a magnetic tape, or a USB stick on which electronically readable control information (e.g., software) is stored. If this control information is read from the data carrier and stored in a computing unit, all the embodiments according to the present embodiments of the methods described above may be carried out. Thus, the present embodiments may also proceed from the computer-readable medium and/or the electronically readable data carrier.

### BRIEF DESCRIPTION OF THE DRAWINGS

Mutually corresponding parts are provided with the same reference characters in all the figures, in which:

FIG. 1 shows one embodiment of a magnetic resonance (MR) scanner and a non-local computing unit;

FIG. 2 shows one embodiment of a method for determining a simulation value describing a safety-relevant variable for an MR measurement;

FIG. 3 shows possible information flows between a computing unit and an MR scanner for performing a method for determining a simulation value describing a safety-relevant variable for an MR measurement; and

FIG. 4 shows an MR pulse sequence including a plurality of RF pulses and gradient pulses.

### DETAILED DESCRIPTION

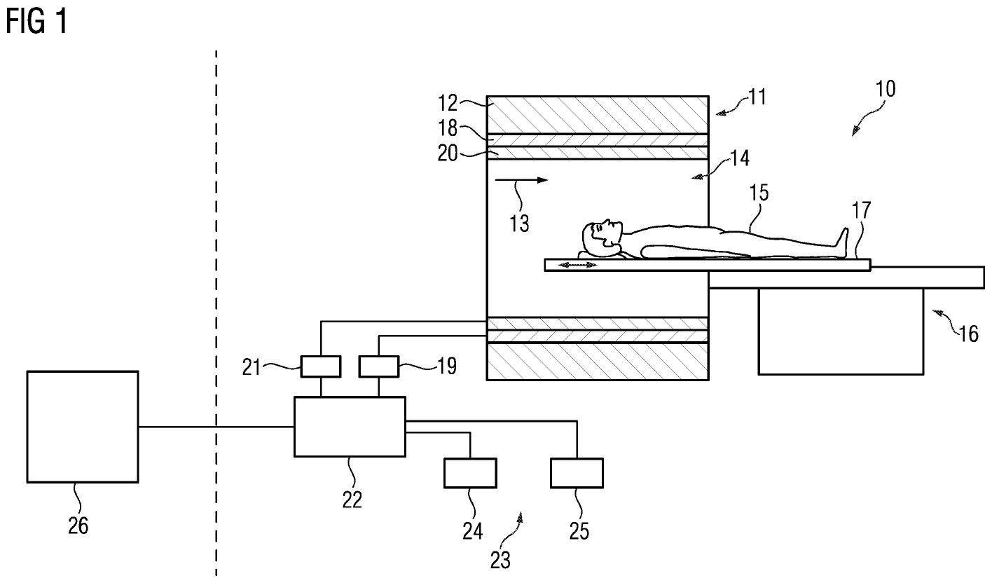

FIG. 1 schematically illustrates one embodiment of a magnetic resonance (MR) scanner 10 and a non-local computing unit 26. The MR scanner 10 includes a magnet unit 11 having a main magnet 12 for generating a powerful and, for example, time-constant main magnetic field 13. In addition, the MR scanner 10 includes a patient tunnel 14 for accommodating a patient 15. In the exemplary embodiment, the patient tunnel 14 is cylindrical in shape and is cylindrically enclosed in a circumferential direction by the magnet unit 11. In principle, however, a different design of the patient tunnel 14 may be provided. The patient 15 may be slid into the patient tunnel 14 by a patient positioning device 16 of the MR scanner 10. For this purpose, the patient positioning device 16 has a patient table 17 that is configured to be movable within the patient tunnel 14.

The magnet unit 11 also includes a gradient coil unit 18 for generating magnetic field gradient pulses (e.g., gradient pulses for short). The gradient pulses are used, for example, for spatial encoding during an MR measurement. The gradient coil unit 18 is controlled by a gradient control unit 19 of the MR scanner 10. The magnet unit 11 also includes a radiofrequency antenna unit 20 that, in this exemplary embodiment, is configured as a body coil integrated in the MR scanner 10 in a fixed manner. The radiofrequency antenna unit 20 is controlled by a radiofrequency antenna control unit 21 of the MR scanner 10, and radiates radiofrequency (RF) pulses into an examination space essentially constituted by a patient tunnel 14 of the MR scanner 10. This causes excitation of atomic nuclei in the main magnetic field 13 generated by the main magnet 12. Magnetic resonance signals are generated by relaxation of the excited atomic nuclei. The radiofrequency antenna unit 20 is configured to receive the magnetic resonance signals.

The MR scanner 10 has a system control unit 22 for controlling the main magnet 12, the gradient control unit 19, and for controlling the radiofrequency antenna control unit 21. The system control unit 22 controls the magnetic resonance device 10 (e.g., for performing an MR pulse sequence). The system control unit 22 also includes an evaluation unit (not shown in more detail) for evaluating the MR signals acquired during an MR measurement. In addition, the MR scanner 10 includes a user interface 23 connected to the system control unit 22. Control information such as MR pulse sequence parameters as well as reconstructed MR images may be displayed on a display unit 24 (e.g., on at least one monitor) of the user interface 23 for medical personnel. The user interface 23 also includes an input unit 25 by which information and/or parameters (e.g., MR pulse sequence parameters) may be entered by medical personnel during a measurement process.

The system control unit 22 of the MR scanner is connected to a computing unit 26. In the present example, the computing unit 26 is a non-local computing unit. The non-local computing unit 26 is separate from the MR scanner 10 and is connected to the MR scanner 10 via a transmission line. However, the computing unit 26 may be a local computing unit. For example, the computing unit 26 may be part of the system control unit 22 of the MR scanner 10.

The computing unit 26 is configured to determine at least one simulation value based on an MR pulse sequence and at least one patient value, and to provide this simulation value to the system control unit 22. In one embodiment, the computing unit 26 may be configured to provide the system control unit 22 with an optimized MR pulse sequence (e.g., MR pulse sequence parameters of an optimized MR pulse sequence).

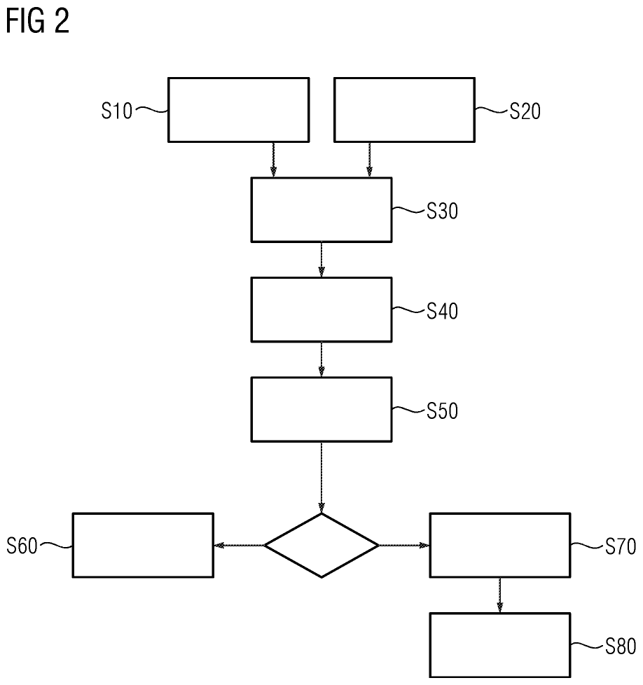

A corresponding method for determining a simulation value describing a safety-relevant variable for an MR measurement is illustrated in FIG. 2. In act S10, an MR pulse sequence is provided to the computing unit 26 by the system control unit 22 and is configured to be used for performing an MR measurement of a patient 15 using an MR scanner 10. The MR pulse sequence includes a temporal succession of a plurality of RF pulses that may be output using the RF antenna unit 20 of the MR scanner 10 and a plurality of gradient pulses that may be output using the gradient coil unit 18 of the MR scanner 10.

In act S20, at least one patient value is provided to the computing unit 26 by the system control unit 22, where the at least one patient value describes a characteristic of the patient 15.

In act S30, at least one simulation value is determined by the computing unit 26 based on the MR pulse sequence and the at least one patient value. The at least one simulation value describes a safety-relevant variable for performing an MR measurement based on the MR pulse sequence, such as a specific absorption rate and/or a gradient stimulation (e.g., a nerve stimulation level). In determining the at least one simulation value in act S30, specific characteristics (e.g., the shape and/or duration and/or amplitude) of the RF pulses (e.g., all the RF pulses) and of the gradient pulses (e.g., all the gradient pulses) of the MR pulse sequence as well as a temporal succession of the RF pulses and the gradient pulses are taken into account. In act S40, the at least one simulation value is provided to the system control unit 22 by the computing unit 26.

In this example, in act S50, the at least one simulation value is compared with a predefined limit value. This comparison may be performed, for example, by the computing unit 26 and/or by the system control unit 22.

If the at least one simulation value does not exceed the predefined limit value, in act S60, an MR measurement of the patient 15 is performed by the MR scanner 10 based on the MR pulse sequence.

For example, it may be provided that in act S70, the MR pulse sequence is optimized based on the simulation value (e.g., by a neural network). In one embodiment, this is done if the at least one simulation value exceeds the predefined limit value. The optimized MR pulse sequence may be provided to the system control unit 22 in act S80.

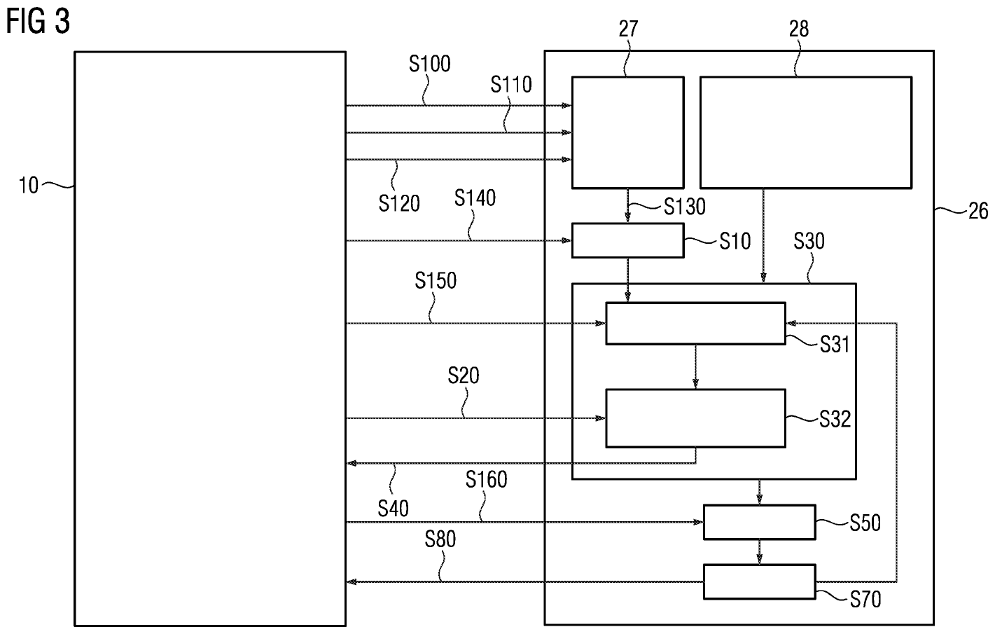

Further possible variants or details are illustrated based on FIG. 3. For example, the computing unit 26 includes, for example, a database 27 containing descriptions of a plurality of MR pulse sequence types. Further descriptions of MR pulse sequence types may be imported into the database in act S100, for example. These may be, for example, MR pulse sequence types developed by any third party (e.g., not by the manufacturer of the MR scanner 10).

In act S110, the MR scanner 10 provides the computing unit with an MR pulse sequence type ID that may be assigned to one MR pulse sequence type of the plurality of MR pulse sequence types stored in the database 27. In addition, in act S120, the MR scanner provides a plurality of MR pulse sequence parameters to the computing unit 26. In act S130, the MR pulse sequence for which at least one simulation value is to be determined may be determined by the computing unit 26 based on the MR pulse sequence type ID and a plurality of MR pulse sequence parameters.

However, in one embodiment, in act S140, the MR pulse sequence is transmitted to the computing unit 26 without recourse to a database 27. The MR pulse sequence may be provided in act S10 by act S130 and/or act S140.

The computing unit 26 may also include a database 28 including at least one pre-calculated auxiliary value. A variation in patient values and/or MR pulse sequence parameters, for example, is assigned to the at least one auxiliary value. The at least one auxiliary value may be based, for example, on modeling of at least one MR pulse sequence type for at least one patient value and/or for at least one MR pulse sequence parameter. In addition, the at least one auxiliary value may take into account a variation in a measurement time for performing the MR measurement. The at least one simulation value may be determined in act S30 using the at least one auxiliary value.

In act S150, the computing unit is provided with at least one adjustment value that the computing unit uses to determine the at least one simulation value in act S31 and/or act S32.

Based on the MR pulse sequence, the MR pulse sequence is unrolled in act S31. Specific characteristics (e.g., the shape and/or duration and/or amplitude) of all the RF pulses and all the gradient pulses of the MR pulse sequence as well as a temporal succession of the RF pulses and the gradient pulses are taken into consideration.

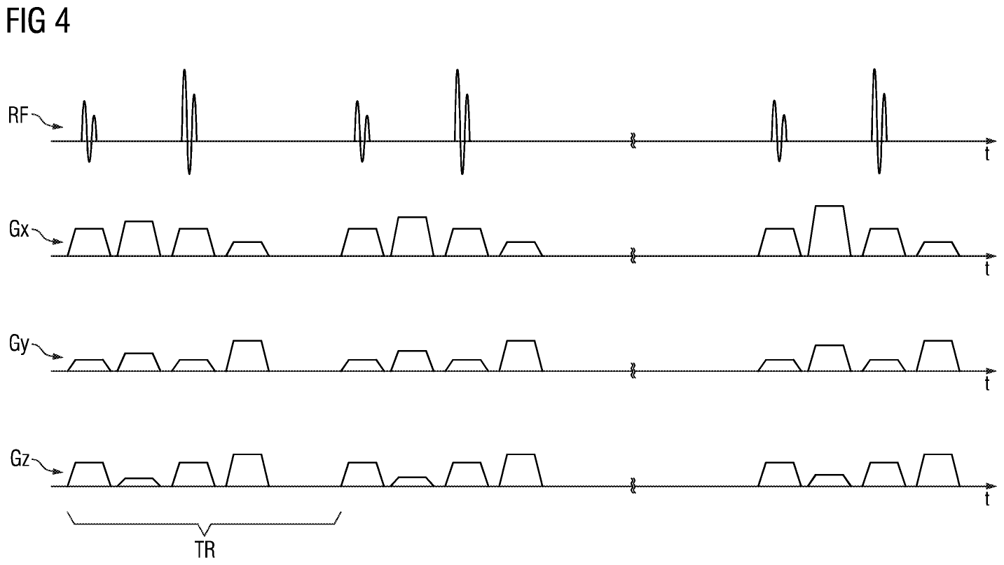

The unrolling of the MR pulse sequence in S31 is shown in FIG. 4, in which a plurality of axes are shown as a function of time t. The MR pulse sequence may be described by a plurality of RF pulses that are shown along the axis RF. These pulses may be output by the RF antenna unit 20 of the MR scanner 10 at particular time intervals. In addition, a plurality of gradient pulses are shown along the Gx, Gy and Gz axes. Gx represents a gradient coil of the gradient coil unit 18 that may generate a magnetic field gradient along an x direction; Gy represents a gradient coil of the gradient coil unit 18 that may generate a magnetic field gradient along a y direction; Gz represents a gradient coil of the gradient coil unit 18 that may generate a magnetic field gradient along a y direction. In one embodiment, x, y, and z form an orthogonal coordinate system. Particular patterns repeat after particular repetition times TR, where, for example, one parameter is varied in each case. For example, an entire slice of the patient 15 may thus be measured successively.

The RF pulses along the axis RF and the gradient pulses along the Gx, Gy, and Gz axes each have specific characteristics (e.g., a specific shape and/or duration and/or amplitude). During the unrolling, all these pulses with their characteristics may be considered (e.g., their effects on the at least one simulation value to be determined are taken into account).

As shown in FIG. 2, the at least one simulation value is determined in act S32 based on the MR pulse sequence unrolled in S31. For this determination, at least one patient value is provided to the computing unit in act S20. The at least one simulation value determined is provided to the MR scanner in act S40. The at least one simulation value may, for example, also be displayed by the display unit 24 of the MR scanner and/or further processed by the system control unit 22.

In act S50, the at least one simulation value may be compared with at least one limit value provided in act S160. In this example, the comparison is performed by the computing unit 26. However, in one embodiment, the comparison may be performed, for example, in the system control unit 22 of the MR scanner.

If necessary, the MR pulse sequence may be optimized in act S70 (e.g., if the comparison shows that the limit value is not met). The optimized MR pulse sequence may be unrolled again in act S31, so that in act S32, a further simulation value is determined for the optimized MR pulse sequence. The further simulation value may be compared with a limit value in act S50 and, if necessary, may be optimized again. A possible optimized sequence may be provided to the MR scanner 10 in act S80.

Due to a possibly high computing outlay, the acts performed in the computing unit 26 may be cloud-based. For example, the descriptions of the plurality of MR pulse sequence types may already be available in the cloud, so that only the protocol parameters set are to be transmitted by the MR scanner 10 in act S120. If necessary, data for a current measurement situation (e.g., adjustment parameters in act S150 or patient parameters, such as height, weight, position and orientation, in act S20) may also be transmitted to the cloud.

The simulation values determined (e.g., prediction values) are then returned from the cloud after the sequences have been completely unrolled there on fast high-performance computers in act S31.

A conceivable enhancement consists in also passing on the corresponding limit values of the variables to be determined in act S160 to the cloud, in order to suggest optimally modified MR pulse sequence parameters for the sequence if a limit is exceeded. More complex optimizations of the settable protocol parameters may take place on the high-performance computers in the cloud, and/or a neural network may be used. For example, using the modified MR pulse sequence parameters, the associated variables to be limited may also be returned as a preview in act S40, which may be displayed using the display unit 24 of the user interface 23 of the MR scanner 10, for example.

For example, to provide the auxiliary values for the database 28, limits for variations in patient and sequence parameters may be pre-calculated and stored. For example, for optimal SAR prediction, each protocol may have already been modeled for a number of patient parameters such as weight and body size, and for optimal stimulation prediction, the slice orientation may have been tilted for a number of angles. In addition, the effect of an increase in measurement time (e.g., increase in averaging or matrix size) may be tested in advance. This procedure allows, for example, real-time prediction of setting changes affecting the executability of protocols during protocol editing.

In a variant, the limit value checking in act S50 takes place, for example, for measurements in the protocol queue that follow the current measurement and are already planned. In this way, possible exceedances may be detected at an early stage, and conflicts may be resolved without “last minute” changes.

In one variant, it is possible to dispense with the use of a cloud. Instead, for example, the system control unit 22 of the MR scanner may also include the computing unit 26 so that the computing unit 26 is integrated locally on the MR scanner.

In another embodiment, no-load periods (e.g., during sampling intervals or overnight) of existing computing units of the MR scanner, such as host or MARS computers, for example, are used to compute models for newly imported MR sequences in advance so that the models are then available later when the MR measurement is started.

The methods and/or devices of the present embodiments may provide the following advantages. All variants of MR pulse sequence types may be covered. Other limitations not otherwise considered (e.g., duty cycle models) may be included without the need to develop special new methods for this purpose. Further, shutdowns due to limit exceedances during scanning may be avoided. In addition, the method allows consistent implementation of safety architectures that do not rely exclusively on online shutdown, but consistently eliminate potential exceedances as early as the commissioning stage; this is advantageous in view of particular, potentially life-threatening consequences (e.g., in the case of active implants such as cardiac pacemakers or even deep brain stimulators). In addition, further counter-proposals for optimized MR pulse sequences (e.g., MR pulse sequence parameters) may be provided during the usual workflow if a currently set MR pulse sequence would result in limits being exceeded.

The methods described in detail above, as well as the computing unit and MR scanner illustrated, are merely exemplary embodiments that may be modified by persons skilled in the art in a wide variety of ways without departing from the scope of the invention. In addition, the use of the indefinite articles “a” or “one” does not exclude the possibility that the features in question may be present more than once. Similarly, the term “unit” does not preclude the components in question from including a plurality of interacting sub-components that may possibly even be spatially distributed.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for determining a simulation value describing a safety-relevant variable for a magnetic resonance (MR) measurement, the method comprising:
providing an MR pulse sequence that is configured to be used for performing an MR measurement of a patient using an MR scanner, wherein the MR pulse sequence comprises a temporal succession of a plurality of RF pulses and a plurality of gradient pulses;
providing at least one patient value, wherein the at least one patient value describes a characteristic of the patient;
determining, by a computing unit, at least one simulation value based on the MR pulse sequence and the at least one patient value, wherein the at least one simulation value describes a safety-relevant variable for performing an MR measurement using the MR pulse sequence, wherein specific characteristics of the plurality of RF pulses and of the plurality of gradient pulses of the MR pulse sequence, and a temporal succession of the plurality of RF pulses and the plurality of gradient pulses are taken into account for determining the at least one simulation value; and
providing the at least one simulation value.

2. The method of claim 1, wherein the specific characteristics of the plurality of RF pulses and of the plurality of gradient pulses include a shape, a duration, an amplitude, or any combination thereof of the plurality of RF pulses and the plurality of gradient pulses.

3. The method of claim 1, wherein the at least one simulation value describes a specific absorption rate, a gradient stimulation, or the specific absorption rate and the gradient stimulation.

4. The method of claim 3, wherein the at least one simulation value describes the gradient stimulation, the gradient stimulation being a nerve stimulation.

5. The method of claim 1, further comprising:
comparing the at least one simulation value with a predefined limit value; and
when the at least one simulation value does not exceed the predefined limit value, performing an MR measurement of the patient using the MR scanner based on the MR pulse sequence.

6. The method of claim 1, wherein determining, by the computing unit, the at least one simulation value comprises determining, by a non-local computing unit, the at least one simulation value.

7. The method of claim 1, wherein the computing unit comprises a database,
wherein the database contains descriptions of a plurality of MR pulse sequence types,
wherein at least one MR pulse sequence type ID is provided to the computing unit,
wherein the at least one MR pulse sequence type ID is assigned to one MR pulse sequence type of the plurality of MR pulse sequence types,
wherein at least one MR pulse sequence parameter is provided to the computing unit, and
wherein the method further comprises determining, by the computing unit, the MR pulse sequence based on the at least one MR pulse sequence type ID and the at least one MR pulse sequence parameter.

8. The method of claim 1, wherein the computing unit comprises a database,
wherein the database comprises at least one pre-calculated auxiliary value, and
wherein the determining of the at least one simulation value takes place using the at least one auxiliary value.

9. The method of claim 8, wherein the at least one auxiliary value is assigned a variation of patient values, MR pulse sequence parameters, or a combination thereof.

10. The method of claim 8, wherein the at least one auxiliary value is based on modeling of at least one MR pulse sequence type for at least one patient value, for at least one MR pulse sequence parameter, or for a combination thereof.

11. The method of claim 8, wherein the at least one auxiliary value takes into consideration a variation in measurement time for performing the MR measurement.

12. The method of claim 1, wherein at least one adjustment value is provided, and
wherein the at least one simulation value is also determined by the computing unit based on the at least one adjustment value.

13. The method of claim 1, wherein a protocol queue with a plurality of MR pulse sequences is provided to the computing unit, and
wherein determining the at least one simulation value comprises determining at least one simulation value for each MR pulse sequence of the plurality of MR pulse sequences.

14. The method of claim 13, further comprising optimizing the MR pulse sequence based on the at least one simulation value.

15. The method of claim 14, wherein optimizing the MR pulse sequence comprises optimizing, by a neural network, the MR pulse sequence.

16. A computing unit configured to determine at least one simulation value based on a magnetic resonance (MR) pulse sequence and at least one patient value, wherein the MR pulse sequence comprises a temporal succession of a plurality of RF pulses and a plurality of gradient pulses, wherein the at least one patient value describes a characteristic of the patient, wherein the at least one simulation value describes a safety-relevant variable for performing an MR measurement based on the MR pulse sequence, the computing unit comprising:
a processor configured determine the at least one simulation value taking into consideration specific characteristics of the plurality of RF pulses and of the plurality of gradient pulses of the MR pulse sequence, and a temporal succession of the plurality of RF pulses and the plurality of gradient pulses.

17. A magnetic resonance (MR) scanner comprising:
a computing unit configured to determine at least one simulation value based on an MR pulse sequence and at least one patient value, wherein the MR pulse sequence comprises a temporal succession of a plurality of RF pulses and a plurality of gradient pulses, wherein the at least one patient value describes a characteristic of the patient,
wherein the at least one simulation value describes a safety-relevant variable for performing an MR measurement based on the MR pulse sequence, the computing unit comprising:
a processor configured determine the at least one simulation value taking into consideration specific characteristics of the plurality of RF pulses and of the plurality of gradient pulses of the MR pulse sequence, and a temporal succession of the plurality of RF pulses and the plurality of gradient pulses
